(19) United States
(12) Patent Application Publication
(10) Pub. No.: US 2010/0016207 A1
(43) Pub. Date: Jan. 21, 2010

(54) Methods and Compositions for Raising Levels and Release of Gamma Aminobutyric Acid

(76) Inventors: Richard J. Wurtman, Boston, MA (US); Lei Wang, Waltham, MA (US)

Correspondence Address: Pearl Cohen Zedek Latzer, LLP, 1500 Broadway, 12th Floor, New York, NY, 10036, US

(21) Appl. No.: 12/084,768

(22) PCT Filed: Nov. 13, 2006
(86) PCT No.: PCT/US06/43988

§ 371 Date: Oct. 6, 2009

Related U.S. Application Data
(60) Provisional application No. 60/735,183, filed on Nov. 10, 2005.

(51) Int. Cl. A61K 38/00; A61K 31/195; A61P 25/28; A61P 25/00

(52) U.S. Cl. 514/2; 514/564

(57) ABSTRACT

The present invention provides methods of increasing the level or release of gamma aminobutyric acid (GABA) in the brain, treating Alzheimer's disease, Huntington's disease, symptoms thereof, anxiety, aggression, insomnia, cognitive or memory disturbances; seizures of any cause (e.g. idiopathic epilepsy), primary or metastatic brain tumors, depression (e.g. bipolar depression), or pain (e.g. neuropathic pain), comprising administration of glutamine, a salt thereof, or a glutamine-rich peptide. The present invention also provides methods for decreasing the level or release of GABA in the brain and treating hepatic encephalopathy, depressed consciousness, and coma, comprising decreasing dietary intake of glutamine.

### FIELD OF INVENTION

The present invention provides methods of increasing the level or release of gamma aminobutyric acid (GABA) in the brain, treating Alzheimer's disease, Huntington's disease, symptoms thereof, anxiety, aggression, insomnia, cognitive or memory disturbances; seizures of any cause (e.g. idiopathic epilepsy), primary or metastatic brain tumors, depression (e.g. bipolar depression), or pain (e.g. neuropathic pain), comprising administration of glutamine, a salt thereof, or a glutamine-rich peptide. The present invention also provides methods for decreasing the level or release of GABA in the brain and treating hepatic encephalopathy, depressed consciousness, and coma, comprising decreasing dietary intake of glutamine.

### BACKGROUND OF THE INVENTION

GABA is the major inhibitory neurotransmitter of the brain, occurring in 30-40% of all synapses (second only to glutamate as a major brain neurotransmitter). The GABA concentration in the brain is 200-1000 times greater than that of the monoamines or acetylcholine. GABA concentrations are decreased in the basal ganglia of Huntington's disease patients, and this deficiency is likely to contribute to the dementia, mood disorders, and psychoses related thereto. Postmortem studies of Alzheimer's patients have shown central GABA deficits, showing the importance of GABA levels in Alzheimer's. In addition, animal studies have shown that increasing GABA levels can inhibit aggression. Thus, methods for increasing GABA levels have multiple applications in many areas of medicine and psychology.

### SUMMARY OF THE INVENTION

The present invention provides methods of increasing the level or release of gamma aminobutyric acid (GABA) in the brain, treating Alzheimer's disease, Huntington's disease, symptoms thereof, anxiety, aggression, insomnia, cognitive or memory disturbances; seizures of any cause (e.g. idiopathic epilepsy), primary or metastatic brain tumors, depression (e.g. bipolar depression), or pain (e.g. neuropathic pain), comprising administration of glutamine, a salt thereof, or a glutamine-rich peptide. The present invention also provides methods for decreasing the level or release of GABA in the brain and treating hepatic encephalopathy, depressed consciousness, and coma, comprising decreasing dietary intake of glutamine.

In one embodiment, the present invention provides a method of increasing a GABA level in a brain of a subject in need thereof, comprising administering to the subject a composition comprising a glutamine, a salt thereof, or a glutamine-rich peptide, thereby increasing a GABA level in a brain of a subject in need thereof.

In another embodiment, the present invention provides a method of increasing or stimulating a release of a GABA in a brain of a subject in need thereof, comprising administering to the subject a composition comprising a glutamine, a salt thereof, or a glutamine-rich peptide, thereby increasing or stimulating a release of a GABA in a brain of a subject in need thereof.

In another embodiment, the present invention provides a method of stimulating a GABA receptor of a neuron in a subject in need thereof, comprising administering to the subject a composition comprising a glutamine, a salt thereof, or a glutamine-rich peptide, thereby stimulating a GABA receptor in a subject in need thereof.

In another embodiment, the present invention provides a method of treating an epilepsy in a subject in need thereof, comprising administering to the subject a composition comprising a glutamine, a salt thereof, or a glutamine-rich peptide, thereby treating epilepsy in a subject in need thereof.

In another embodiment, the present invention provides a method for treating an Alzheimer's disease in a subject in need thereof, comprising administering to the subject a composition comprising a glutamine, a salt thereof, or a glutamine-rich peptide, thereby treating an Alzheimer's disease in a subject in need thereof.

In another embodiment, the present invention provides a method for treating a Huntington's disease or a dementia, mood disorder, or psychosis resulting therefrom, in a subject in need thereof, comprising administering to the subject a composition comprising a glutamine, a salt thereof, or a glutamine-rich peptide, thereby treating a Huntington's disease or a dementia, mood disorder, or psychosis resulting therefrom, in a subject in need thereof.

In another embodiment, the present invention provides a method of treating an anxiety disorder or decreasing an incidence of aggression in a subject in need thereof, comprising administering to the subject a composition comprising a glutamine, a salt thereof, or a glutamine-rich peptide, thereby treating an anxiety disorder or decreasing an incidence of aggression in a subject in need thereof.

In another embodiment, the present invention provides a method of treating an insomnia or producing sedation in a subject in need thereof, comprising administering to the subject a composition comprising a glutamine, a salt thereof, or a glutamine-rich peptide, thereby treating an insomnia or producing sedation in a subject in need thereof.

In another embodiment, the present invention provides a method of decreasing a GABA level in a brain of a subject in need thereof, comprising decreasing the dietary intake of glutamine by the subject, thereby decreasing a GABA level in a brain of a subject in need thereof.

In another embodiment, the present invention provides a method of decreasing a release of a GABA in a brain of a subject in need thereof, comprising decreasing the dietary intake of glutamine by the subject, thereby decreasing a release of a GABA in a brain of a subject in need thereof.

In another embodiment, the present invention provides a method of treating a depressed consciousness or coma in a subject in need thereof, comprising decreasing the dietary intake of glutamine by the subject, thereby treating a depressed consciousness or coma in a subject in need thereof.

In another embodiment, the present invention provides a method of treating a hepatic encephalopathy in a subject in need thereof, comprising decreasing the dietary intake of glutamine by the subject, thereby treating a hepatic encephalopathy in a subject in need thereof.

In another embodiment, the present invention provides a method of decreasing stimulation of a GABA receptor of a neuron in a subject in need thereof, comprising decreasing a dietary intake of glutamine by said subject, thereby decreasing stimulation of a GABA receptor in a subject in need thereof

### BRIEF DESCRIPTION OF THE FIGURES

FIG. 1. Effect of oral glutamine administration on brain (striatal) GABA levels. *=p<0.05.

FIG. 2: Relationship between striatal glutamine and GABA levels. Data were analyzed by linear regression.

FIG. 3. Effect of oral glutamine (Gln) administration on brain (striatal) glutamate and glutamine levels. “*” denotes p<0.05.

FIG. 4. Effect of oral glutamine administration on plasma glutamate and glutamine levels.

FIG. 5. Schematic depiction of NMDA administration timeline.

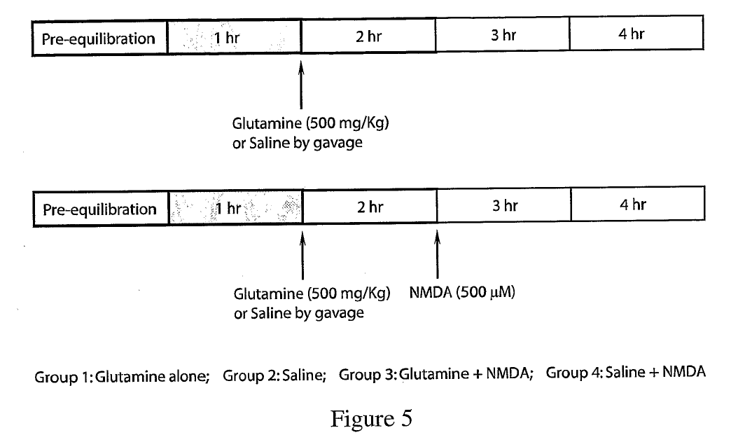

FIG. 6. Spontaneous GABA release measured by microdialysis in rat striatum after glutamine administration by gavage. *p<0.05 compared to saline.

FIG. 7. Spontaneous and evoked GABA release measured by microdialysis in rat striatum after glutamine administration by gavage. *=p<0.05 compared to saline.

### DETAILED DESCRIPTION OF THE INVENTION

The present invention provides methods of increasing the level or release of gamma aminobutyric acid (GABA) in the brain, treating Alzheimer's disease, Huntington's disease, symptoms thereof, anxiety, aggression, insomnia, cognitive or memory disturbances; seizures of any cause (e.g. idiopathic epilepsy), primary or metastatic brain tumors, depression (e.g. bipolar depression), or pain (e.g. neuropathic pain), comprising administration of glutamine, a salt thereof, or a glutamine-rich peptide. The present invention also provides methods for decreasing the level or release of GABA in the brain and treating hepatic encephalopathy, depressed consciousness, and coma, comprising decreasing dietary intake of glutamine.

In one embodiment, the present invention provides a method of increasing a GABA level in a brain of a subject in need thereof, comprising administering to the subject a composition comprising a glutamine, thereby increasing a GABA level in a brain of a subject in need thereof. In another embodiment, the present invention provides a method of increasing a GABA level in a brain of a subject in need thereof, comprising administering to the subject a salt of glutamine, thereby increasing a GABA level in a brain of a subject in need thereof. In another embodiment, the present invention provides a method of increasing a GABA level in a brain of a subject in need thereof, comprising administering to the subject a composition comprising a glutamine-rich peptide, thereby increasing a GABA level in a brain of a subject in need thereof.

In another embodiment, the GABA level that is modulated by methods of the present invention is a striatal GABA level (Examples). In another embodiment, the GABA level is the GABA level in the hippocampus. In another embodiment, the GABA level is in the cerebral cortex. In another embodiment, the GABA level is in the hypothalamus. In another embodiment, the GABA level is in the thalamus. In another embodiment, the GABA level is in the brainstem. In another embodiment, the GABA level is in the cerebellum. In another embodiment, the GABA level is in any other brain region that is known to have GABA-releasing neurons. Each possibility represents a separate embodiment of the present invention.

In another embodiment, the method of administration of glutamine in methods of the present invention is oral administration. In another embodiment, the oral administration comprises increasing the dietary level of glutamine. In another embodiment, the method of administration is any method of administration enumerated below. In another embodiment, the method of administration is any other method of administration known in the art. Each possibility represents a separate embodiment of the present invention.

In another embodiment, the present invention provides a method of increasing or stimulating a release of a GABA in a brain of a subject in need thereof, comprising administering to the subject a composition comprising a glutamine, thereby increasing or stimulating a release of a GABA in a brain of a subject in need thereof. In another embodiment, the present invention provides a method of increasing or stimulating a release of a GABA in a brain of a subject in need thereof, comprising administering to the subject a composition comprising a glutamine salt, thereby increasing or stimulating a release of a GABA in a brain of a subject in need thereof. In another embodiment, the present invention provides a method of increasing or stimulating a release of a GABA in a brain of a subject in need thereof, comprising administering to the subject a composition comprising a glutamine-rich peptide, thereby increasing or stimulating a release of a GABA in a brain of a subject in need thereof.

In another embodiment, the release is a release is in the striatum, as exemplified herein. In another embodiment, the release is in the hippocampus. In another embodiment, the release is in the cerebral cortex. In another embodiment, the release is in the hypothalamus. In another embodiment, the release is in the thalamus. In another embodiment, the release is in the brainstem. In another embodiment, the release is in the cerebellum. In another embodiment, the release is in any other brain region that is known to have GABA-releasing neurons. Each possibility represents a separate embodiment of the present invention.

As provided herein, GABA levels and release in the brain, both spontaneous and glutamate-evoked, are increased by Gln administration. Under the conditions utilized herein, NMDA-evoked GABA release, is a surrogate for glutamate-evoked release. As is known in the art, NMDA acts on the glutamate receptor. Findings of the present invention in the stratum are, in another embodiment, applicable to any brain region that contains GABA-releasing neurons, e.g. the regions enumerated above.

In another embodiment, the release that is increased, stimulated, or decreased by a method of the present invention is a spontaneous or basal release. In another embodiment, the release is a stimulated release. Each possibility represents a separate embodiment of the present invention.

In another embodiment, the stimulated release is stimulated by a neurotransmitter that functions upstream of GABA. In another embodiment, the stimulated release is stimulated by an agonist of a neurotransmitter receptor (e.g. NMDA). In another embodiment, the neurotransmitter is any other neurotransmitter known in the art. Each possibility represents a separate embodiment of the present invention.

In another embodiment, the present invention provides a method of stimulating a GABA receptor of a neuron in a subject in need thereof, comprising administering to the subject a composition comprising a glutamine, thereby stimulating a GABA receptor in a subject in need thereof. In another embodiment, the present invention provides a method of stimulating a GABA receptor of a neuron in a subject in need thereof, comprising administering to the subject a composition comprising a glutamine salt, thereby stimulating a GABA receptor in a subject in need thereof. In another embodiment, the present invention provides a method of stimulating a GABA receptor of a neuron in a subject in need thereof, comprising administering to the subject a composition comprising a glutamine-rich peptide, thereby stimulating a GABA receptor in a subject in need thereof.

In another embodiment, the GABA receptor is a GABA-A receptor. In another embodiment, the GABA receptor is a GABA-B receptor. In another embodiment, the GABA receptor is any other GABA receptor known in the art. Each possibility represents a separate embodiment of the present invention.

The neuron that is the target of methods of the present invention is, in another embodiment, a striatal neuron. In another embodiment, the neuron is a GABAergic neuron (e.g. a projecting GABAergic neuron, pallido-subthalamic GABAergic neuron, striatopallidal GABA neuron, or a GABAergic efferent neuron). In another embodiment, the neuron is a striatopallidal GABAergic neuron (e.g. a dorsal GABAergic striatopallidal neuron or a ventral GABAergic striatopallidal neuron). In another embodiment, the neuron is any other GABA receptor-expressing neuron known in the art. In another embodiment, the neuron is any other GABA-secreting neuron known in the art.

In another embodiment, the present invention provides a method of treating an epilepsy in a subject in need thereof, comprising administering to the subject a composition comprising a glutamine, thereby treating epilepsy in a subject in need thereof. In another embodiment, the present invention provides a method of treating an epilepsy in a subject in need thereof, comprising administering to the subject a composition comprising a glutamine salt, thereby treating epilepsy in a subject in need thereof. In another embodiment, the present invention provides a method of treating an epilepsy in a subject in need thereof, comprising administering to the subject a composition comprising a glutamine-rich peptide, thereby treating epilepsy in a subject in need thereof. Epilepsy is associated with depressed GABA levels; thus, the present invention shows that administering gln treats epilepsy.

In another embodiment, the target neuron of methods of the present invention is a nucleus reticularis of the thalamus (NRT) neuron. In another embodiment, the neuron is a thalamic neuron (e.g. a thalamic relay neuron). Dysfunction or deficient GABA receptor signaling in each of these types of neurons, among others enumerated below, plays a role in epileptic seizures and other neurological disorders. Thus, methods of the present invention have utility in treating and preventing epilepsy and other neurological disorders by modulating GABA signaling in these and other neurons.

In another embodiment, the neuron is a motor neuron. In another embodiment, the neuron is an interneuron. In another embodiment, the neuron is a sensory neuron. In another embodiment, the neuron is a preganglionic neuron. In another embodiment, the neuron is a GABAergic neuron of any type. In another embodiment, the neuron is a peptidergic neuron. In another embodiment, the neuron is a postganglionic neuron. In another embodiment, the neuron is a cholinergic neuron. In another embodiment, the neuron is a noradrenergic neuron. In another embodiment, the neuron is a cortical neuron. In another embodiment, the neuron is a cerebellar neuron. In another embodiment, the neuron is a hippocampal neuron. In another embodiment, the neuron is a dopaminergic neuron. In another embodiment, the neuron is a striatonigral neuron. In another embodiment, the neuron is a striatoentopeduncular neuron. In another embodiment, the neuron is a glutamatergic neuron. In another embodiment, the neuron is a striatonigral-striatoentopeduncular neuron. In another embodiment, the neuron is a hypothalamic neuron. In another embodiment, the neuron is a brainstem neuron. Each type of neuron represents a separate embodiment of the present invention.

In another embodiment, the neuron whose GABA receptor activity is modulated by methods of the present invention is in the central nervous system (CNS). Findings of the present invention are applicable to any GABA-releasing neurons, whether in the brain or elsewhere in the CNS. Each possibility represents a separate embodiment of the present invention.

The epilepsy that is treated by a method of the present invention, is, in another embodiment, a partial-onset epilepsy. In another embodiment, the epilepsy is a generalized-onset epilepsy. In another embodiment, the epilepsy is an idiopathic epilepsy. In another embodiment, the epilepsy is a frontal lobe epilepsy. In another embodiment, the epilepsy is associated with Lennox-Gastaut Syndrome. In another embodiment, the epilepsy is an early myoclonic encephalopathy. In another embodiment, the epilepsy is a benign childhood epilepsy. In another embodiment, the epilepsy is a juvenile myoclonic epilepsy. In another embodiment, the epilepsy is an epileptic encephalopathy. In another embodiment, the epilepsy is an epileptiform encephalopathy. In another embodiment, the epilepsy is a posttraumatic epilepsy. In another embodiment, the epilepsy is a temporal lobe epilepsy. In another embodiment, the epilepsy is a reflex epilepsy. In another embodiment, the epilepsy is Epilepsia Partialis Continua. In another embodiment, the epilepsy is Status Epilepticus. In another embodiment, the epilepsy is any other type of epilepsy known in the art.

In another embodiment, the epilepsy comprises partial-onset seizures. In another embodiment, the epilepsy comprises generalized-onset seizures. In another embodiment, the epilepsy comprises simple partial seizures. In another embodiment, the epilepsy comprises complex partial seizures. In another embodiment, the epilepsy comprises secondarily generalized seizures. In another embodiment, the epilepsy comprises tonic-clonic seizures. In another embodiment, the epilepsy comprises absence seizures. In another embodiment, the epilepsy comprises pseudo seizures. In another embodiment, the epilepsy comprises shuddering attacks. In another embodiment, the epilepsy comprises febrile seizures. In another embodiment, the epileptic seizure is caused by a primary or metastatic brain tumor. In another embodiment, the epileptic seizure is caused by another space-occupying lesion (e.g. a blood clot). In another embodiment, the epilepsy comprises any other type of seizure known in the art. Each type of epilepsy represents a separate embodiment of the present invention.

In another embodiment, the present invention provides a method for treating an Alzheimer's disease in a subject in need thereof, comprising administering to the subject a composition comprising a glutamine, thereby treating an Alzheimer's disease in a subject in need thereof. In another embodiment, the present invention provides a method for treating an Alzheimer's disease in a subject in need thereof, comprising administering to the subject a composition comprising a glutamine salt, thereby treating an Alzheimer's disease in a subject in need thereof. In another embodiment, the present invention provides a method for treating an Alzheimer's disease in a subject in need thereof, comprising administering to the subject a composition comprising a glutamine-rich peptide, thereby treating an Alzheimer's disease in a subject in need thereof. Alzheimer's disease is associated with dysfunctional and/or deficient GABA-receptor signaling in brain neurons. Thus, methods of the present invention, which increase GABA-receptor signaling, have utility in treating Alzheimer's disease.

In another embodiment, the Alzheimer's disease is at an early stage. In another embodiment, the Alzheimer's disease is at a mild stage. In another embodiment, the Alzheimer's disease is at a moderate stage. In another embodiment, the Alzheimer's disease is at a late stage. In another embodiment, the Alzheimer's disease is at a severe stage. In another embodiment, the Alzheimer's disease is at an undetermined stage. In another embodiment, the Alzheimer's disease is at any stage of the disease known in the art. Each stage represents a separate embodiment of the present invention.

Methods for diagnosing Alzheimer's disease are well known in the art. In another embodiment, the stage of Alzheimer's disease is assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) becomes manifestin demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulla is observed. In stage 7 (severe Alzheimer's disease), speech ability becomes limited to just a few words and intelligible vocabulary may be limited to a single word. Patient loses the ability to walk, sit up, smile and eventually cannot hold up the head. Each stage of Alzheimer's disease represents a separate embodiment of the present invention.

In another embodiment, the present invention provides a method for treating a Huntington's disease or a dementia, mood disorder, or psychosis resulting therefrom, in a subject in need thereof, comprising administering to the subject a composition comprising a glutamine, thereby treating a Huntington's disease or a dementia, mood disorder, or psychosis resulting therefrom, in a subject in need thereof. In another embodiment, the present invention provides a method for treating a Huntington's disease or a dementia, mood disorder, or psychosis resulting therefrom, in a subject in need thereof, comprising administering to the subject a composition comprising a glutamine salt, thereby treating a Huntington's disease or a dementia, mood disorder, or psychosis resulting therefrom, in a subject in need thereof. In another embodiment, the present invention provides a method for treating a Huntington's disease or a dementia, mood disorder, or psychosis resulting therefrom, in a subject in need thereof, comprising administering to the subject a composition comprising a glutamine-rich peptide, thereby treating a Huntington's disease or a dementia, mood disorder, or psychosis resulting therefrom, in a subject in need thereof. Huntington's disease is associated with dysfunctional and/or deficient GABA-receptor signaling in brain neurons. Thus, methods of the present invention, which increase GABA-receptor signaling, have utility in treating Huntington's disease.

In another embodiment, the present invention provides a method of treating an anxiety disorder or decreasing an incidence of aggression in a subject in need thereof, comprising administering to the subject a glutamine, thereby treating an anxiety disorder or decreasing an incidence of aggression in a subject in need thereof. In another embodiment, the present invention provides a method of treating an anxiety disorder or decreasing an incidence of aggression in a subject in need thereof, comprising administering to the subject a composition comprising a glutamine salt, thereby treating an anxiety disorder or decreasing an incidence of aggression in a subject in need thereof. In another embodiment, the present invention provides a method of treating an anxiety disorder or decreasing an incidence of aggression in a subject in need thereof, comprising administering to the subject a composition comprising a glutamine-rich peptide, thereby treating an anxiety disorder or decreasing an incidence of aggression in a subject in need thereof. These disorders are associated with dysfunctional and/or deficient GABA-receptor signaling in brain neurons. Thus, methods of the present invention, which increase GABA-receptor signaling, have utility in treating these disorders.

In another embodiment, the present invention provides a method of treating an insomnia or producing sedation in a subject in need thereof, comprising administering to the subject a composition comprising a glutamine, thereby treating an insomnia or producing sedation in a subject in need thereof. In another embodiment, the present invention provides a method of treating an insomnia or producing sedation in a subject in need thereof, comprising administering to the subject a composition comprising a glutamine salt, thereby treating an insomnia or producing sedation in a subject in need thereof. In another embodiment, the present invention provides a method of treating an insomnia or producing sedation in a subject in need thereof, comprising administering to the subject a composition comprising a glutamine-rich peptide, thereby treating an insomnia or producing sedation in a subject in need thereof. These disorders are associated with dysfunctional and/or deficient GABA-receptor signaling in brain neurons. Thus, methods of the present invention, which increase GABA-receptor signaling, have utility in treating these disorders.

In another embodiment, the present invention provides a method of treating a cognitive disturbance in a subject in need thereof, comprising administering to the subject a composition comprising a glutamine, a salt thereof, or a glutamine-rich peptide, thereby treating a cognitive disturbance in a subject in need thereof. In another embodiment, the present invention provides a method of treating a memory disturbance in a subject in need thereof, comprising administering to the subject a composition comprising a glutamine, a salt thereof, or a glutamine-rich peptide, thereby treating a memory disturbance in a subject in need thereof. These disorders are associated with dysfunctional and/or deficient GABA-receptor signaling in brain neurons. Thus, methods of the present invention, which increase GABA-receptor signaling, have utility in treating these disorders.

In another embodiment, the present invention provides a method of ameliorating a brain tumor in a subject in need thereof, comprising administering to the subject a composition comprising a glutamine, a salt thereof, or a glutamine-rich peptide, thereby ameliorating a brain tumor in a subject in need thereof. In another embodiment, the tumor is a primary tumor. In another embodiment, the tumor is a metastatic tumor. These disorders are associated with dysfunctional and/or deficient GABA-receptor signaling in brain neurons. Thus, methods of the present invention, which increase GABA-receptor signaling, have utility in treating these disorders.

As provided herein, levels and release of GABA in the brain can be increased by increasing dietary intake of glutamine. Many drugs currently used for facilitating sleep or producing sedation (e.g. Ambien, lorazepam, Librium, and valium) work by increasing GABA-mediated transmission. Thus, the findings of the present invention show that increasing dietary intake of glutamine has utility in treating facilitating sleep and producing sedation.

In another embodiment, the present invention provides a method of treating in a subject in need thereof another disorder associated with dysfunctional and/or deficient GABA levels or release, comprising administering to the subject a composition comprising a glutamine, thereby treating the disorder in a subject in need thereof. In another embodiment, the present invention provides a method of treating such a disorder in a subject in need thereof, comprising administering to the subject a composition comprising a glutamine salt, thereby treating the disorder in a subject in need thereof. In another embodiment, the present invention provides a method of treating such a disorder in a subject in need thereof, comprising administering to the subject a composition comprising a glutaime-rich peptide, thereby treating the disorder in a subject in need thereof.

In another embodiment, the disorder associated with dysfunctional and/or deficient GABA levels or release is a disorder for which Neurontin® (gabapentin) or a related medication has been shown to be effective. In other embodiments, the disorder is Reflex Sympathetic Dystrophy (RSD), brain injury, essential tremors, sleep dysfunction, Interstitial Cystitis, refractory GU tract pain, agitation secondary to dementia, muscle cramps, inflammatory injuries, tinnitus, phantom limb pain, cocaine dependence, TMJ, neuropathic pain, Shoulder-Hand Syndrome, hemifacial spasms, peripheral neuropathy; pain, nystagmus, and spasticity of Multiple Sclerosis (MS); trigeminal neuralgia, prophylaxis and for acute migraines, for pain secondary to epidural fibrosis, acute and postherpetic neuralgia (Shingles), acute pain from Herpes Simplex, post-operative pain, myofascial pain (MPS), radiation myelopathy, cancer pain, Restless Leg Syndrome (RLS), Lou Gehrig's Disease (ALS), Periodic Leg Movement (PLM), chronic pain, Bipolar Disorder, social phobias, somatiform pain with depression, mood disorders, situational depression, diabetic neuropathy or clinical depression. Each of the above disorder has been successfully treated with Neurontin or a related medication. In another embodiment, the disorder is any other disorder that is treatable with Neurontin or a related medication. Each possibility represents a separate embodiment of the present invention.

In another embodiment, the disorder associated with dysfunctional and/or deficient GABA levels or release is a disorder for which benzodiazepine drugs (e.g. Xanax® (Alprazolam) or Klonopin®) have been shown to be effective. In other embodiments, the disorder is social anxiety disorder (social phobia), panic disorder, or symptoms of generalized anxiety disorder, adjustment disorders, mood disorders, or psychotic disorders. Each of the above disorder has been successfully treated with benzodiazepine drugs. In another embodiment, the disorder is any other disorder that is treatable with a benzodiazepine drug. Each possibility represents a separate embodiment of the present invention.

In another embodiment, the disorder associated with dysfunctional and/or deficient GABA levels or release is a disorder for which barbiturate drugs (e.g. Butalbital and Phenobarbital) have been shown to be effective. In other embodiments, the disorder is allergic rhinitis (AR) or cyclic vomiting syndrome (CVS). Each of the above disorder has been successfully treated with barbiturate drugs. In another embodiment, the disorder is any other disorder that is treatable with a barbiturate drug. Each possibility represents a separate embodiment of the present invention.

In another embodiment, a glutamine-containing composition utilized in a method of the present invention further comprises pyridoxine (vitamin B6). The chow used in the findings of the present invention contained a vitamin mix that included pyridoxine. The presence of pyridoxine in the body is required, in another embodiment, for the conversion of Gln to Glu. In another embodiment, pyridoxine is required for conversion of Glu to GABA. Thus, in these embodiments, supplementation of pyridoxine further increases GABA levels, release, and the therapeutic effects thereof. In another embodiment, a composition of the present invention further comprises another vitamin that is required for conversion of Gln to Glu. In another embodiment, the composition further comprises another vitamin that is required for conversion of Gln to Glu. In these embodiments, the vitamin supplementation further increases GABA levels, release, and the therapeutic effects thereof. Each possibility represents a separate embodiment of the present invention.

In another embodiment, methods of the present invention comprise administration of a compound that breaks down or is metabolized in the body to glutamine. In another embodiment, the compound is a peptides or proteins rich in glutamine. In another embodiment, the compound is a synthetic di- or tri-peptide comprising glutamine. In another embodiment, the methods comprise administration of a glutamine salt. In another embodiment, the methods comprise administration of a compound related to glutamine, as described herein. Each possibility represents a separate embodiment of the present invention.

In another embodiment, the present invention provides a use of glutamine in the manufacture of a medicament, pharmaceutical composition, or nutritional supplement for treating 1 of the above diseases. In another embodiment, the present invention provides a use of a glutamine salt in the manufacture of a medicament, pharmaceutical composition, or nutritional supplement for treating 1 of the above diseases. In another embodiment, the present invention provides a use of a glutamine-rich peptide in the manufacture of a medicament, pharmaceutical composition, or nutritional supplement for treating 1 of the above diseases. In another embodiment, the medicament, pharmaceutical composition, or nutritional supplement further comprises 1 of the active compounds or substances enumerated above. Each possibility represents a separate embodiment of the present invention.

In another embodiment, the present invention provides a composition comprising glutamine for treating 1 of the above diseases. In another embodiment, the present invention provides a composition comprising a glutamine salt for treating 1 of the above diseases. In another embodiment, the present invention provides a composition comprising a glutamine-rich peptide for treating 1 of the above diseases. In another embodiment, the composition further comprises 1 of the active compounds or substances enumerated above. Each possibility represents a separate embodiment of the present invention.

In another embodiment, the present invention provides a method of decreasing a GABA level in a brain of a subject in need thereof, comprising decreasing the dietary intake of glutamine by the subject, thereby decreasing a GABA level in a brain of a subject in need thereof.

In another embodiment, brain GABA levels, release or signaling are decreased by administration of other AA that compete with blood glutamine for transport into the brain (the large neutral amino acids (LNAA; leucine/isoleucine/valine/tyrosine/phenylalanine), as shown by the findings of the present invention. In another embodiment, methods of present invention that treat consequences of overactive GABA signaling comprise administration of LNAA. In another embodiment, methods of present invention comprise the step of contacting a subject with a compound or composition that suppresses glutamine transport across the BBB. In another embodiment, the compound or composition is comprises 1 or several LNAA. Each possibility represents a separate embodiment of the present invention.

In another embodiment, the present invention provides a method of decreasing a release of a GABA in a brain of a subject in need thereof, comprising decreasing the dietary intake of glutamine by the subject, thereby decreasing a release of a GABA in a brain of a subject in need thereof. As provided herein, findings of the present invention have shown that dietary intake of glutamine modulates the level and release of GABA in the brain. Thus, reducing dietary intake of glutamine reduces the level and release of GABA in the brain.

In another embodiment, the present invention provides a method of treating a depressed consciousness or coma in a subject in need thereof, comprising decreasing the dietary intake of glutamine by the subject, thereby treating a depressed consciousness or coma in a subject in need thereof.

Administration of benzodiazepines and barbiturates to patients with cirrhosis increases GABA-ergic tone and predisposes patients to depressed consciousness. In addition, flumazenil (a benzodiazepine antagonist) reverses hepatic encephalopathy in patients with cirrhosis. Thus, methods of the present invention that decrease GABA-receptor signaling have utility in treating depressed consciousness.

In another embodiment, the depressed consciousness or coma is associated with a liver cirrhosis. In another embodiment, the depressed consciousness or coma is associated with any other cause of depressed consciousness or coma known in the art. Each possibility represents a separate embodiment of the present invention.

In another embodiment, the present invention provides a method of treating a hepatic encephalopathy in a subject in need thereof, comprising decreasing the dietary intake of glutamine by the subject, thereby treating a hepatic encephalopathy in a subject in need thereof. Hepatic encephalopathy is associated with hyperactive and/or uncontrolled GABA-receptor signaling in brain neurons. Thus, methods of the present invention, which decrease GABA-receptor signaling, have utility in treating hepatic encephalopathy.

As provided herein, GABA synthesis and release in rat striatum are increased by increasing dietary glutamine (Gln) levels. In another embodiment, the increase is due to increased circulating Gln levels. In another embodiment, the increase is due to increased glutamate (Glu) levels. In another embodiment, the increased Glu levels are increased brain Glu levels. In another embodiment, the increased Glu levels CNS Glu levels. Each possibility represents a separate embodiment of the present invention.

In another embodiment, the present invention provides a method of decreasing stimulation of a GABA receptor of a neuron in a subject in need thereof, comprising decreasing a dietary intake of glutamine by said subject, thereby decreasing stimulation of a GABA receptor in a subject in need thereof.

Gamma-aminobutyric acid (GABA) is, in another embodiment, an inhibiting neurotransmitter in the brain. In another embodiment, GABA binds to 2 major classes of receptors: GABA-A and GABA-B. Each possibility represents a separate embodiment of the present invention. Hepatic encephalopathy is a syndrome observed in some patients with cirrhosis that is marked by personality changes, intellectual impairment, and a depressed level of consciousness. The diversion of portal blood into the systemic circulation appears to be a prerequisite for the syndrome. In another embodiment, hepatic encephalopathy develops in patients who do not have cirrhosis who undergo portocaval shunt surgery.

Methods for diagnosing hepatic encephalopathy are well known in the art. In another embodiment, symptoms are graded on the following scale: Grade 0—Subclinical; normal mental status, but minimal changes in memory, concentration, intellectual function, coordination. Grade 1—Mild confusion, euphoria or depression, decreased attention, slowing of ability to perform mental tasks, irritability, disorder of sleep pattern (i.e. inverted sleep cycle). Grade 2—Drowsiness, lethargy, gross deficits in ability to perform mental tasks, obvious personality changes, inappropriate behavior, intermittent disorientation (usually for time). Grade 3—Somnolent but arousable, unable to perform mental tasks, disorientation to time and place, marked confusion, amnesia, occasional fits of rage, speech is present but incomprehensible. Grade 4—Coma, with or without response to painful stimuli.

In another embodiment, elevated arterial or free venous serum ammonia level is the classic laboratory abnormality reported in patients with hepatic encephalopathy. In another embodiment, hepatic encephalopathy is detected by classic but nonspecific electroencephalogram (EEG) changes of high-amplitude low-frequency waves and triphasic waves. In another embodiment, hepatic encephalopathy is detected by intracranial lesions (e.g. subdural hematoma, intracranial bleeding, cerebrovascular accident, tumor, and abscess). In another embodiment, hepatic encephalopathy is diagnosed by any other method known in the art. Each possibility represents a separate embodiment of the present invention.

In another embodiment, the present invention provides a use of an LNAA or mixture of several LNAA in the manufacture of a medicament, pharmaceutical composition, or nutritional supplement for treating 1 of the above diseases. In another embodiment, the medicament, pharmaceutical composition, or nutritional supplement further comprises 1 of the active compounds or substances enumerated above. Each possibility represents a separate embodiment of the present invention.

In another embodiment, the present invention provides a composition comprising an LNAA or mixture of several LNAA for treating 1 of the above diseases. In another embodiment, the composition further comprises 1 of the active compounds or substances enumerated above. Each possibility represents a separate embodiment of the present invention.

Various embodiments of dosage ranges of glutamine and related compounds are contemplated by this invention. In another embodiment, the dosage is 20 mg per day. In another embodiment, the dosage is 10 mg/day. In another embodiment, the dosage is 30 mg/day. In another embodiment, the dosage is 40 mg/day. In another embodiment, the dosage is 60 mg/day. In another embodiment, the dosage is 80 mg/day. In another embodiment, the dosage is 100 mg/day. In another embodiment, the dosage is 150 mg/day. In another embodiment, the dosage is 200 mg/day. In another embodiment, the dosage is 300 mg/day. In another embodiment, the dosage is 400 mg/day. In another embodiment, the dosage is 600 mg/day. In another embodiment, the dosage is 800 mg/day. In another embodiment, the dosage is 1 g/day. In another embodiment, the dosage is 1.5 g/day. In another embodiment, the dosage is 2 g/day. In another embodiment, the dosage is 3 g/day. In another embodiment, the dosage is 3 g/day. In another embodiment, the dosage is 5 g/day. In another embodiment, the dosage is 6 g/day. In another embodiment, the dosage is 8 g/day. In another embodiment, the dosage is 10 g/day. In another embodiment, the dosage is more than 10 g/day.

In another embodiment, the dosage is 10 mg/dose. In another embodiment, the dosage is 30 mg/dose. In another embodiment, the dosage is 40 mg/dose. In another embodiment, the dosage is 60 mg/dose. In another embodiment, the dosage is 80 mg/dose. In another embodiment, the dosage is 100 mg/dose. In another embodiment, the dosage is 150 mg/dose. In another embodiment, the dosage is 200 mg/dose. In another embodiment, the dosage is 300 mg/dose. In another embodiment, the dosage is 400 mg/dose. In another embodiment, the dosage is 600 mg/dose. In another embodiment, the dosage is 800 mg/dose. In another embodiment, the dosage is 1000 mg/dose. In another embodiment, the dosage is 1500 mg/dose. In another embodiment, the dosage is 2000 mg/dose.

In another embodiment, the dosage is 10-20 mg/dose. In another embodiment, the dosage is 20-30 mg/dose. In another embodiment, the dosage is 20-40 mg/dose. In another embodiment, the dosage is 30-60 mg/dose. In another embodiment, the dosage is 40-80 mg/dose. In another embodiment, the dosage is 50-100 mg/dose. In another embodiment, the dosage is 50-150 mg/dose. In another embodiment, the dosage is 100-200 mg/dose. In another embodiment, the dosage is 200-300 mg/dose. In another embodiment, the dosage is 300-400 mg/dose. In another embodiment, the dosage is 400-600 mg/dose. In another embodiment, the dosage is 500-800 mg/dose. In another embodiment, the dosage is 800-1000 mg/dose. In another embodiment, the dosage is 1000-1500 mg/dose. In another embodiment, the dosage is 1500-2000 mg/dose.

Each of the above doses represents a separate embodiment of the present invention.

In another embodiment, the present invention provides a composition for treating one of the above diseases, disorders, or conditions, the composition comprising glutamine, a salt thereof, or a source thereof.

In another embodiment, a treatment protocol of the present invention is therapeutic. In another embodiment, the protocol is prophylactic. Each possibility represents a separate embodiment of the present invention.

In other embodiments, the present invention provides a method of treating any disease, disorder, symptom, or side effect associated with Alzheimer's disease, Huntington's disease, or encephalopathy. In other embodiments, the present invention provides a method of preventing Alzheimer's disease, Huntington's disease, encephalopathy, or any disease, disorder, symptom, or side effect associated therewith. In other embodiments, the present invention provides a method of reducing an incidence of Alzheimer's disease, Huntington's disease, encephalopathy, or any disease, disorder, symptom, or side effect associated therewith. Each disease, disorder, symptom, or side effect represents a separate embodiment of the present invention.

In another embodiment, the neurological disease being treated is a chronic disease, and administration of a composition comprising a glutamine stimulates or enhances GABA levels or release over the long term. In another embodiment, the composition comprising glutamine is administered throughout the course of disease. In another embodiment, the composition comprising glutamine is administered during symptomatic stages of the disease. In another embodiment, the composition comprising glutamine is administered as a pretreatment for prevention of the disease. In another embodiment, the composition comprising glutamine is administered as a post-treatment for preventing relapse of the disease. Each possibility represents a separate embodiment of the present invention.

In another embodiment, the present invention relates to the use of glutamine and/or its analog, derivative, isomer, metabolite, pharmaceutically acceptable salt, pharmaceutical product, hydrate, N-oxide, or a combination thereof for treating, preventing, suppressing, inhibiting or reducing the incidence of any of the above diseases, disorders, conditions, or symptoms. In another embodiment, the methods of the present invention comprise administering an analog of the glutamine. In another embodiment, the methods of the present invention comprise administering a derivative of the glutamine. In another embodiment, the methods of the present invention comprise administering an isomer of the glutamine. In another embodiment, the methods of the present invention comprise administering a metabolite of the glutamine. In another embodiment, the methods of the present invention comprise administering a pharmaceutically acceptable salt of the glutamine. In another embodiment, the methods of the present invention comprise administering a pharmaceutical product of the glutamine. In another embodiment, the methods of the present invention comprise administering a hydrate of the glutamine. In another embodiment, the methods of the present invention comprise administering an N-oxide of the glutamine. In another embodiment, the methods of the present invention comprise administering any of a combination of an analog, derivative, isomer, metabolite, pharmaceutically acceptable salt, pharmaceutical product, hydrate or N-oxide of the glutamine.

As defined herein, the term “isomer” includes, but is not limited to, optical isomers and analogs, structural isomers and analogs, conformational isomers and analogs, and the like.

The invention includes “pharmaceutically acceptable salts” of amino-substituted compounds with organic and inorganic acids, for example, citric acid and hydrochloric acid. The invention also includes N-oxides of the amino substituents of glutamine. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes, in another embodiment, derivatives of glutamine. The term “derivatives” includes, in one embodiment, ether derivatives, acid derivatives, amide derivatives, ester derivatives and the like. In another embodiment, this invention further includes hydrates of glutamine. The term “hydrate” includes, in one embodiment, hemihydrate, monohydrate, dihydrate, trihydrate and the like.

### Pharmaceutical Compositions and Methods of Administration

In one embodiment, the methods of the present invention comprise administering a pharmaceutical composition comprising glutamine and/or its analog, derivative, isomer, metabolite, pharmaceutically acceptable salt, pharmaceutical product, hydrate, N-oxide, or any combination thereof; and a pharmaceutically acceptable carrier. The pharmaceutical composition is administered to a male subject any of the above conditions.

“Pharmaceutical composition” refers, in one embodiment, to a therapeutically effective amount of the active ingredient, i.e. glutamine, together with a pharmaceutically acceptable carrier or diluent. A “therapeutically effective amount” refers, in one embodiment, to that amount which provides a therapeutic effect for a given condition and administration regimen.

The pharmaceutical compositions containing glutamine can be, in other embodiments, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the pharmaceutical compositions are administered by intravenous, intra-arterial, or intramuscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intramuscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, glutamine or its physiologically tolerated derivatives such as salts, esters, N-oxides, and the like are prepared and applied as solutions, suspensions, or emulsions in a physiologically acceptable diluent with or without a pharmaceutical carrier.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of glutamine over a period of time.

In another embodiment, the active compound is delivered in a vesicle, e.g. a liposome.

As used herein “pharmaceutically acceptable carriers or diluents” are well known to those skilled in the art. The carrier or diluent is, in various embodiments, a solid carrier or diluent for solid formulations, a liquid carrier or diluent for liquid formulations, or mixtures thereof.

In another embodiment, solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations can be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In other embodiments, the compositions further comprises binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e. compositions in which glutamine is released over a period of time after administration. Controlled- or sustained-release compositions include formulation in lipophilic depots (e.g. fatty acids, waxes, oils). In another embodiment, the composition is an immediate-release composition, i.e. a composition in which all of the glutamine is released immediately after administration.

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions that contain an active component, for example by mixing, granulating, or tablet-forming processes, is well understood in the art. The active therapeutic ingredient is often mixed with excipients that are pharmaceutically acceptable and compatible with the active ingredient. For oral administration, glutamine or its physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, glutamine or its physiologically tolerated derivatives such as salts, esters, N-oxides, and the like are converted into a solution, suspension, or emulsion, if desired with the substances customary and suitable for this purpose, for example, solubilizers or other substances.

An active component is, in another embodiment, formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Each of the above additives, excipients, formulations and methods of administration represents a separate embodiment of the present invention.

In one embodiment, the methods of the present invention comprise administering glutamine as the sole active ingredient. In another embodiment, glutamine is administered in combination with one or more therapeutic agents. These agents are appropriate for the disease or disorder that is being treated, as is well known in the art.

### EXPERIMENTAL DETAILS SECTION

### Example 1

### Oral Glutamine Administration Increases Brain GABA Levels

### Materials and Experimental Methods

### Animal Care

Male adult Sprague Dawley (SD) rats (˜250 g) (n=9) were reared under and exposed to a 12 hr light/dark cycle, and provided with food (16% protein) and water ad libitum. Gln (0.5 grams per kilogram (g/Kg) or 1.0 g/Kg, in saline) or saline were administered by gavage. Rats were sacrificed by decapitation 2.5 hours (h) later.

### Tissue Neurotransmitter Contents

To measure GABA contents, brains were quickly dissected on a chilled dissection board. Striata were homogenized with HClO₄ and neutralized with 0.1 M Borate buffer (1:10). Data were normalized by the wet weights of the samples.

### Neurotransmitter Analysis

GABA was measured by HPLC, using an ODS column and OPA-sulfite derivation with electrochemical detection. OPA-sulfite HPLC-EC derivation was used to measure GABA levels in both brain tissue and microdialysis samples. The samples were derivatized at 4° C. to allow usage of an auto-sampler (Alltech 580). The detector used was an ESA Coulochem® II 5100A with an ESA microdialysis cell.

### Data Analysis

Data are presented as mean+/−S.E.M. ANOVA or t-test was used to determine differences between groups (significance level, p<0.05).

### Results

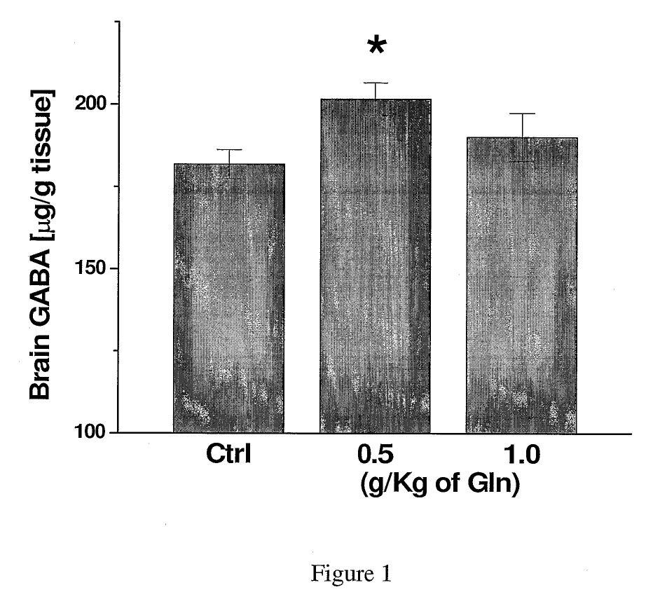

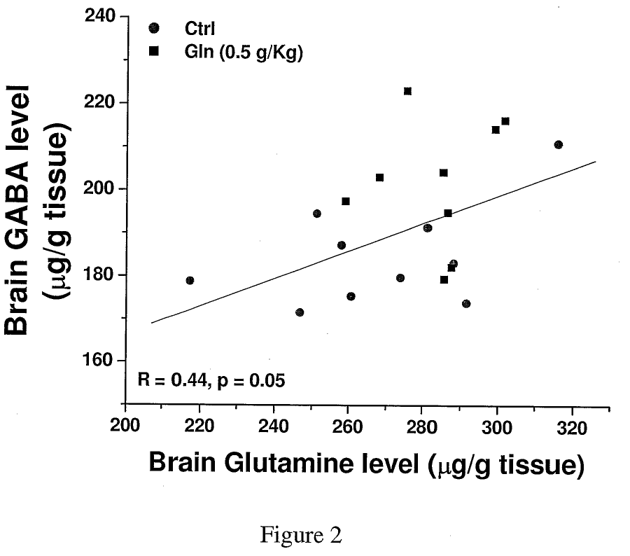

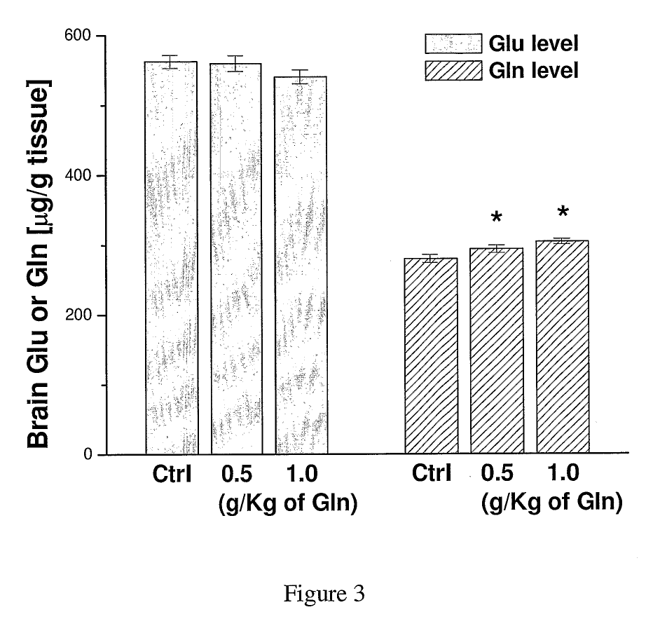

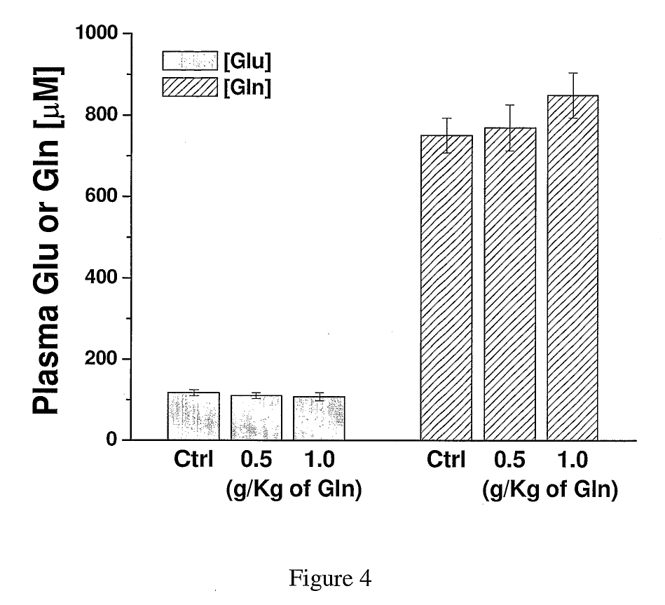

To assess the effect of glutamine (Gln) administration on brain GABA levels, rats were administered either saline or Gln (0.5 g/kg, body weight) by gavage. Striata were collected 2.5 hrs after dosing, and GABA levels were determined. Gin significantly (p<0.05) increased striatal GABA levels, from 182±4 (n=11) to 202±4 mg/g. (FIG. 1). When data from individual animals was analyzed by linear regression, a significant relationship was observed between striatal glutamine and GABA levels (FIG. 2). Straital Gln and glutamate (Glu) levels were determined as well. Gin administration significantly increased striatal Gln levels and slightly decreased striatal Glu levels (FIG. 3). In plasma, Gln increased plasma Gln levels, but not Glu levels (FIG. 4).

These results demonstrate that GABA synthesis in the brain is increased by Gln administration.

### Example 2

### Glutamine Administration Increases Spontaneous and NMDA-evoked Brain GABA Release

### Materials and Experimental Methods

### Microdialysis

Microdialysis was carried out in freely moving rats. Rats were anesthetized, and CMA/11 guide cannulas were permanently implanted into right striatum (AP=+0.5, ML=−3.0 from Bregma, DV=−3.3 mm from Dura) 1 week prior to microdialysis sampling. Dialysis collection was performed using a CMA/11 probe (4 mm), perfused with artificial cerebrospinal fluid (aCSF) at 1.5 ml/min and collected at 20 min intervals.

### NMDA Administration

GABAergic neurons were stimulated by NMDA (500 μM) in aCSF, 1 hr after Gln administration by gavage.

### Results

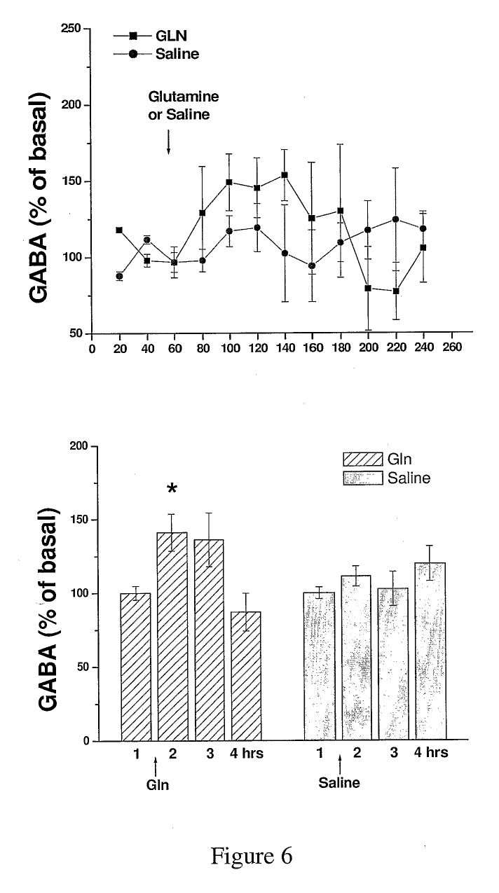

To determine the effect of Gln administration on spontaneous GABA release, in vivo microdialysis was also carried out in striata of rats treated as described in Example 1. Maximal spontaneous GABA release was significantly increased by Gln administration (141±12 vs. 111±7%; FIG. 6).

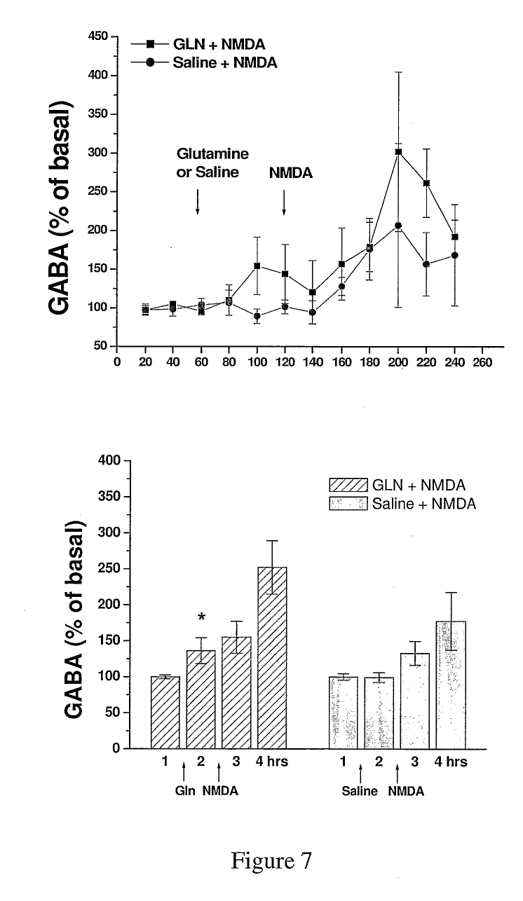

In addition, NMDA-evoked GABA release was increased (252±37 vs. 177±40%; FIG. 7).

Thus, striatal GABA levels and release in the brain, both spontaneous and NMDA-evoked, are increased by Gln administration. Accordingly, Gln administration (e.g. by increasing dietary Gln levels) is an effective treatment for disorders and symptoms characterized or accompanied by deficiencies in brain GABA levels or release.

What is claimed is:

1. A method of increasing or stimulating a gamma aminobutyric acid (GABA) level in a brain of a subject in need thereof, comprising administering to said subject a composition comprising a glutamine, a salt thereof, or a glutamine-rich peptide, thereby increasing a GABA level in a brain of a subject in need thereof.

2. The method of claim 1, wherein said GABA level is a striatal GABA level.

3. The method of claim 1, wherein said administering is orally administering.

4. A method of increasing or stimulating a release of a gamma aminobutyric acid (GABA) in a brain of a subject in need thereof, comprising administering to said subject a composition comprising a glutamine, a salt thereof, or a glutamine-rich peptide, thereby increasing or stimulating a release of a GABA in a brain of a subject in need thereof.

5. The method of claim 4, wherein said release is in the striatum.

6. The method of claim 4, wherein said release is a spontaneous or basal release.

7. The method of claim 4, wherein said release is a glutamate-evoked release.

8. The method of claim 4, wherein said administering is orally administering.

9. A method of stimulating a gamma aminobutyric acid (GABA) receptor of a neuron in a subject in need thereof, comprising administering to said subject a composition comprising a glutamine, a salt thereof, or a glutamine-rich peptide, thereby stimulating a GABA receptor in a subject in need thereof.

10. The method of claim 9, wherein said GABA receptor is a GABA-A receptor.

11. The method of claim 9, wherein said GABA receptor is a GABA-B receptor.

12. The method of claim 9, wherein said administering is orally administering.

13. The method of claim 9, wherein said neuron is a nucleus reticularis of the thalamus (NRT) neuron.

14. The method of claim 9, wherein said neuron is a thalamic relay neuron.

15. A method of treating an epilepsy in a subject in need thereof, comprising administering to said subject a composition comprising a glutamine, a salt thereof, or a glutamine-rich peptide, thereby treating epilepsy in a subject in need thereof.

16. The method of claim 15, wherein said epilepsy is a partial-onset epilepsy.

17. The method of claim 15, wherein said epilepsy comprises generalized-onset seizures.

18. The method of claim 15, wherein said administering is orally administering.

19. A method for treating an Alzheimer's disease in a subject in need thereof, comprising administering to said subject a composition comprising a glutamine, a salt thereof, or a glutamine-rich peptide, thereby treating an Alzheimer's disease in a subject in need thereof.

20. The method of claim 19, wherein said administering is orally administering.

21. A method for treating a Huntington's disease or a dementia, mood disorder, or psychosis resulting therefrom in a subject in need thereof, comprising administering to said subject a composition comprising a glutamine, a salt thereof, or a glutamine-rich peptide, thereby treating a Huntington's disease or a dementia, mood disorder, or psychosis resulting therefrom in a subject in need thereof.

22. The method of claim 21, wherein said administering is orally administering.

23. A method of treating an anxiety disorder or decreasing an incidence of aggression in a subject in need thereof, comprising administering to said subject a composition comprising a glutamine, a salt thereof, or a glutamine-rich peptide, thereby treating an anxiety disorder or decreasing an incidence of aggression in a subject in need thereof.

24. The method of claim 23, wherein said administering is orally administering.

25. A method of treating an insomnia or producing sedation in a subject in need thereof, comprising administering to said subject a composition comprising a glutamine, a salt thereof, or a glutamine-rich peptide, thereby treating an insomnia or producing sedation in a subject in need thereof.

26. The method of claim 25, wherein said administering is orally administering.

27. A method of decreasing a gamma aminobutyric acid (GABA) level in a brain of a subject in need thereof, comprising decreasing a dietary intake of glutamine by said subject, or contacting said subject with a compound or composition that suppresses glutamine transport across the blood-brain barrier, thereby decreasing a GABA level in a brain of a subject in need thereof.

28. The method of claim 27, wherein said GABA level is a striatal GABA level.

29. A method of decreasing a release of a gamma aminobutyric acid (GABA) in a brain of a subject in need thereof, comprising decreasing a dietary intake of glutamine by said subject, or contacting said subject with a compound or composition that suppresses glutamine transport across the blood-brain barrier, thereby decreasing a release of a GABA in a brain of a subject in need thereof.

30. The method of claim 29, wherein said release is in the striatum.

31. The method of claim 29, wherein said release is a spontaneous or basal release.

32. The method of claim 29, wherein said release is a glutamate-evoked release.

33. A method of treating a depressed consciousness or coma in a subject in need thereof, comprising decreasing a dietary intake of glutamine by said subject, or contacting said subject with a compound or composition that suppresses glutamine transport across the blood-brain barrier, thereby treating a depressed consciousness or coma in a subject in need thereof.

34. The method of claim 33, wherein said depressed consciousness or coma is associated with a liver cirrhosis.

35. A method of treating a hepatic encephalopathy in a subject in need thereof, comprising decreasing a dietary intake of glutamine by said subject, or contacting said subject with a compound or composition that suppresses glutamine transport across the blood-brain barrier, thereby treating a hepatic encephalopathy in a subject in need thereof.

36. A method of decreasing stimulation of a gamma aminobutyric acid (GABA) receptor of a neuron in a subject in need thereof, comprising decreasing a dietary intake of glutamine by said subject, or contacting said subject with a compound or composition that suppresses glutamine transport across the blood-brain barrier, thereby decreasing stimulation of a GABA receptor in a subject in need thereof.

37. The method of claim 36, wherein said GABA receptor is a GABA-A receptor.

38. The method of claim 36, wherein said GABA receptor is a GABA-B receptor.

39. The method of claim 36, wherein said neuron is a nucleus reticularis of the thalamus (NRT) neuron.

40. The method of claim 36, wherein said neuron is a thalamic relay neuron.
